(19) United States
(12) Patent Application Publication
(10) Pub. No.: US 2017/0056057 A1
(43) Pub. Date: Mar. 2, 2017

(54) SYSTEM AND METHOD FOR ULTRASONICALLY SENSING AND ABLATING TISSUE

(71) Applicant: VytronUS, Inc., Sunnyvale, CA (US)

(72) Inventors: Hira V. Thapliyal, Los Altos, CA (US); David A. Gallup, Alameda, CA (US); James A. Arenson, Woodside, CA (US); Danielo B. Piazza, Livermore, CA (US); Patrick Phillips, Los Altos, CA (US); Hidenori Shikata, Sunnyvale, CA (US)

(73) Assignee: VytronUS, Inc., Sunnyvale, CA (US)

(21) Appl. No.: 15/349,887

(22) Filed: Nov. 11, 2016

Related U.S. Application Data
(63) Continuation-in-part of application No. 12/695,857, filed on Jan. 28, 2010.
Application No. 12/695,857 is a continuation-in-part of application No. 11/747,862, filed on May 11, 2007, now Pat. No. 7,950,397.
(60) Provisional application No. 61/148,809, filed on Jan. 30, 2009.
(60) Provisional application No. 60/747,137, filed on May 12, 2006.
(60) Provisional application No. 60/919,831, filed on Mar. 23, 2007.

(51) Int. Cl. A61B 17/32; A61B 8/00; A61B 8/08; A61B 8/12

(52) U.S. Cl. CPC A61B 17/320068; A61B 8/12; A61B 8/54; A61B 8/463; A61B 8/5223; A61B 2017/320072; A61B 2017/00106

(57) ABSTRACT

A method of mapping tissue includes sensing a first region and a second region of a chamber of body tissue. The sensing includes moving an ultrasound transducer of a catheter over a surface of the region along a sensing pattern, and using the ultrasound transducer to gather a set of echo-anatomical data in an amplitude mode at a plurality of points along the sensing pattern. The set of echo-anatomical data comprises distances between the ultrasound transducer and the surface at the plurality of points. A three-dimensional surface map is generated using the set of echo-anatomical data from each region. The surface maps of the regions are combined to form a combined surface map. Methods also include using a set of echo-anatomical data to generate a three-dimensional surface map of a region, from a detected border of the body tissue and detected motion phases of the region.

### CROSS-REFERENCES TO RELATED APPLICATIONS

The present application is a continuation-in-part of U.S. patent application Ser. No. 12/695,857, filed on Jan. 28, 2010 and entitled “System and Method for Ultrasonically Sensing and Ablating Tissue”; which is a non-provisional of, and claims the benefit of U.S. Provisional Patent Application No. 61/148,809 filed Jan. 30, 2009, the entire contents of which are incorporated herein by reference. U.S. patent application Ser. No. 12/695,857 is also a continuation-in-part of U.S. patent application Ser. No. 11/747,862, now U.S. Pat. No. 7,950,397 filed on May 11, 2007; which is a non-provisional of and claims the benefit of U.S. Provisional Patent Application No. 60/747,137 filed May 12, 2006, and 60/919,831 filed Mar. 23, 2007, the entire contents of which are incorporated herein by reference.

The present application is also related to the following: U.S. Pat. No. 7,942,871; U.S. Pat. No. 9,155,588; U.S. Patent Publication No. 2009/0312693; U.S. Patent Publication No. 2010/0152582; U.S. Patent Publication No. 2009/0312673; U.S. Patent Publication No. 2010/0049099; U.S. Patent Publication No. 2010/0016762; U.S. Pat. No. 8,475,379; U.S. Pat. No. 9,033,885; U.S. Pat. No. 8,414,508; U.S. Pat. No. 9,192,789; U.S. Provisional Patent Application No. 61/254,997; and U.S. Patent Publication No. 2011/0257563. The entire contents of each of the above is incorporated herein by reference.

### BACKGROUND

The present application generally relates to systems and methods for creating ablation zones in human tissue. More specifically, the present application relates to the treatment of atrial fibrillation (‘AF’) of the heart by using ultrasound energy. While the present application emphasizes treatment of atrial fibrillation, one of skill in the art will appreciate that this is not intended to be limiting, and that the systems and methods disclosed herein may also be used to treat other tissues and conditions, including other arrhythmias like ventricular fibrillation.

The condition of atrial fibrillation is characterized by the abnormal (usually very rapid) beating of the left atrium of the heart which is out of synch with the normal synchronous movement (normal sinus rhythm) of the heart muscle. In normal sinus rhythm, the electrical impulses originate in the sino-atrial node (‘SA node’) which resides in the right atrium. The abnormal beating of the atrial heart muscle is known as ‘fibrillation’ and is caused by electrical impulses originating instead at points other than the SA node, for example, in the pulmonary veins (PVs).

There are pharmacological treatments for this condition with varying degrees of success. In addition, there are surgical interventions aimed at removing the aberrant electrical pathways from PV to the left atrium (‘LA’) such as the ‘Cox-Maze III Procedure’. This procedure has been shown to be 99% effective but requires special surgical skills and is time consuming. Thus, there has been considerable effort to copy the Cox-Maze procedure using a less invasive percutaneous catheter-based approach. Less invasive treatments have been developed which involve use of some form of energy to ablate (or kill) the tissue surrounding the aberrant focal point where the abnormal signals originate in PV. The most common methodology is the use of radio-frequency (‘RF’) electrical energy to heat the muscle tissue and thereby ablate it. The aberrant electrical impulses are then prevented from traveling from PV to the atrium (achieving the ‘conduction block’) and thus avoiding the fibrillation of the atrial muscle. Other energy sources, such as microwave, laser, and ultrasound have been utilized to achieve the conduction block. In addition, techniques such as cryoablation, administration of ethanol, and the like have also been used. Some of these methods and devices are described below.

There has been considerable effort in developing catheter based systems for the treatment of AF using radiofrequency (RF) energy. One such method includes a catheter having proximal and distal electrodes at the catheter tip. The catheter can be bent in a coil shape, and positioned inside a pulmonary vein. The tissue of the inner wall of the PV is then ablated in an attempt to kill the source of the aberrant heart activity.

Another source used in ablation is microwave energy. One such intraoperative device consists of a probe with a malleable antenna which has the ability to ablate the atrial tissue.

Still another catheter based method utilizes the cryogenic technique where the tissue of the atrium is frozen below a temperature of −60 degrees C. This results in killing of the tissue in the vicinity of the PV thereby eliminating the pathway for the aberrant signals causing the AF. Cryo-based techniques have also been a part of the partial Maze procedures described above. More recently, Dr. Cox and his group have used cryoprobes (cryo-Maze) to duplicate the essentials of the Cox-Maze III procedure.

Other recent approaches for the treatment of AF involve the use of ultrasound energy. The target tissue of the region surrounding the pulmonary vein is heated with ultrasound energy emitted by one or more ultrasound transducers. One such approach includes a catheter distal tip portion equipped with a balloon and containing an ultrasound element. The balloon serves as an anchoring means to secure the tip of the catheter in the pulmonary vein. The balloon portion of the catheter is positioned in the selected pulmonary vein and the balloon is inflated with a fluid which is transparent to ultrasound energy. The transducer emits the ultrasound energy which travels to the target tissue in or near the pulmonary vein and ablates it. The intended therapy is to destroy the electrical conduction path around a pulmonary vein and thereby restore the normal sinus rhythm. The therapy involves the creation of a multiplicity of lesions around individual pulmonary veins as required.

Yet another catheter device using ultrasound energy includes a catheter having a tip with an array of ultrasound elements in a grid pattern for the purpose of creating a three-dimensional image of the target tissue. An ablating ultrasound transducer is provided which is in the shape of a ring which encircles the imaging grid. The ablating transducer emits a ring of ultrasound energy at 10 MHz frequency.

In many of the above approaches, the devices and systems involve the ablation of tissue inside a pulmonary vein or of the tissue at the location of the ostium. This may require complex positioning and guiding of the treatment devices to the target site. The ablation is achieved by means of contact between the device and the tissue. Also, many of these systems often require a catheter to be repositioned multiple times within the heart in order to map the atrium or other chamber. Repositioning may require complex manipulation of the catheter and thus this process can be cumbersome. The repositioning also requires imaging to assist in the complex manipulation, where common imaging techniques that are utilized are 2D fluoroscopy and 3D electric-anatomical mapping.

Other ablation systems may be used to map tissue surfaces. For example, one commercially available system uses a high energy focused ultrasound (HIFU) catheter to capture two-dimensional images of a prostate gland relating to blood flow in the target tissue. The user then manually marks tissue components on the individual 2-dimensional images. Thereafter, the images are formed into a three-dimensional model, and a chosen area is ablated in a pinpoint manner. A table, which maps transducer parameters to expected lesion size, is employed to aid in ablation. During the process, the transducer must be repeatably positioned at the same location in order for the method to be effectively carried out. While promising, this system is not optimized for ablation of cardiac tissue.

In the cardiac field methods exist for treating cardiac arrhythmias with no discrete target. A description of the heart chamber anatomy, such as the physical dimensions of the chamber, is obtained and an activation map of a patient's heart is created using locatable catheters. A conduction velocity map is derived from the activation map. Then, a refractory period map is acquired. Appropriate values from the conduction velocity map and the refractory period map are used to create a dimension map, which is then analyzed to determine ablation lines or points.

### SUMMARY

The present application generally relates to systems and methods for creating ablation zones in human tissue. More specifically, the present application relates to the treatment of atrial fibrillation of the heart using ultrasound energy. While the present application emphasizes treatment of atrial fibrillation, one of skill in the art will appreciate that this is not intended to be limiting, and that the systems and methods disclosed herein may also be used to treat other arrhythmias such as ventricular fibrillation, as well as other tissues and conditions.

A method of mapping tissue includes sensing a first region and a second region of a chamber of body tissue with a catheter, the catheter comprising an ultrasound transducer. The sensing includes moving the ultrasound transducer of the catheter over a surface of the region along a sensing pattern, and using the ultrasound transducer to gather a set of echo-anatomical data in an amplitude mode at a plurality of points along the sensing pattern. The set of echo-anatomical data comprises distances between the ultrasound transducer and the surface of the body tissue at the plurality of points. A three-dimensional surface map is generated using the set of echo-anatomical data from each region. The surface maps of the regions are combined to form a combined surface map.

In some embodiments, the methods include sensing a region of a chamber of body tissue with a catheter, the catheter comprising an ultrasound transducer and electromagnetic sensors. The sensing includes moving the ultrasound transducer of the catheter over a surface of the region along a sensing pattern, and using the ultrasound transducer to gather a set of echo-anatomical data in an amplitude mode at a plurality of points along the sensing pattern. The set of echo-anatomical data includes distances between the ultrasound transducer and the surface at the plurality of points. The set of echo-anatomical data is used to detect a border of the body tissue for each point in the plurality of points, and also to detect motion phases of the chamber of body tissue. The border may be at least one of an epicardial border and an endocardial border. A three-dimensional surface map of the region is generated, being derived from the detected border of the body tissue and the detected motion phases of the region. The method also includes displaying the three-dimensional surface map.

In some aspects, a method for echo-anatomically mapping tissue comprises advancing a catheter toward a target treatment tissue. The catheter comprises a proximal end, a distal end, an ultrasound transducer adjacent the distal end, and a console adjacent the proximal end. The console is configured to control movement of the catheter, and the ultrasound transducer is configured to sense the target treatment tissue. A first region of the target treatment tissue is sensed with the ultrasound transducer while moving the ultrasound transducer along a first sensing pattern. A first 3-dimensional surface map of the first region is generated. A second region of the target treatment tissue is sensed with the ultrasound transducer while moving the ultrasound transducer along a second sensing pattern. A second 3-dimensional surface map of the second region is generated. The first and the second 3-dimensional surface maps are combined to form a combined surface map.

The advancing step may comprise percutaneously introducing the catheter into vasculature of a patient and transseptally passing the catheter through an atrial septal wall of the patient's heart into a left atrium. Sensing of the first or the second region may comprise operating the transducer in amplitude mode (A-mode). The first or the second sensing pattern may comprise a raster pattern or a spiral pattern. Sensing of the first or the second regions may also comprise delivering a beam of ultrasound energy from the transducer to the target treatment tissue. The sensing of the first or the second regions may be performed without establishing direct contact between the transducer and the tissue. The first sensed region may be the same or different than the second sensed region. The first sensing pattern may be the same or different than the second sensing pattern.

Generating the first or the second 3-dimensional surface map may comprise visually displaying the combined surface map.

The method may further comprise identifying anatomical features in the first sensed region or the second sensed region. The anatomical features in the first or the second region may comprise one or more pulmonary veins. The identifying step may comprise capturing data indicating distance between the transducer and the target treatment tissue at a plurality of points along the first or the second sensing patterns.

The method may also comprise ablating the target treatment tissue with the ultrasound transducer while moving the ultrasound transducer along a first ablation path. The first ablation path may form a lesion around the identified anatomical features. The lesion may block aberrant electrical pathways in the tissue so as to reduce or eliminate atrial fibrillation. The ablating step may comprise selecting the first ablation path from a catalog of available lesion paths based on the identified anatomical features. The first ablation path may be automatically selected from the catalog of available lesion paths, or a physician may prescribe the first ablation path. The method may further comprise accepting or rejecting the selected ablation path by a physician or other operator. A physician or other operator may also modify the selected ablation path. The catalog of available lesion paths may be stored on a memory element coupled to the console. The method may further comprise adding, deleting, or modifying lesion paths stored on the memory element. The ablating may be performed without establishing direct contact between the transducer and the tissue. The method may comprise drawing the first ablation path by a physician or other operator, or the first ablation path may be suggested by the console.

The method may further comprise visually displaying the combined surface map. The method may also comprise superimposing the first ablation path on the combined surface map, and the resulting superimposed map may be visually displayed. The method may further comprise monitoring deviations from the selected lesion path during the ablating. The ablating may be corrected so as to minimize deviations from the selected lesion path. The correction may comprise moving the transducer. Sensing of the first or the second region may also be synchronized with a patient's the cardiac cycle. The method may further comprise determining lesion thickness along the first ablation path.

These and other embodiments are described in further detail in the following description related to the appended drawing figures.

### BRIEF DESCRIPTION OF THE DRAWINGS

FIG. 1 schematically illustrates an example catheter system for ultrasonically sensing and ablating cardiac tissue.

FIGS. 2A-2D illustrate example sensing patterns.

FIGS. 3A-3F illustrate example 3D maps for six neighboring portions of the atrial tissue surface.

FIG. 3G illustrates a 3D map obtained by combining the six maps of FIGS. 3A-3F.

FIGS. 4A-4J illustrate example lesion paths.

FIGS. 5A-5B illustrate example ablation lesions.

FIGS. 6A-6E illustrate example pattern parameters for sensing patterns.

FIG. 7 illustrates an example 3D anatomical map.

FIGS. 8A-8D illustrate a family of motion maps.

FIG. 9 illustrates an example tissue thickness map.

FIG. 10 is a flow chart of generating 3D maps in accordance with the present embodiments.

FIG. 11 is a flow chart of motion, thickness, and static maps in accordance with the present embodiments.

### DETAILED DESCRIPTION

Overview. The present disclosure emphasizes, but is not limited to catheter systems and methods for ultrasonically sensing and ablating tissue to treat atrial fibrillation. A catheter equipped with an ultrasonic transducer is used to sense and scan at least some portion of atrial heart tissue surface. The ultrasonically sensed data is then used to generate a 3-dimensional (3D) echo-anatomical map of the tissue surface, where maps of scanned regions can be assembled into a combined surface map. In some embodiments, one or more anatomical features are utilized to form the combined surface map; in other embodiments, one or more anatomical features are identified based on the generated 3-dimensional map. The anatomical features may then be electrically isolated using tissue ablation. In one embodiment, the anatomical features are pulmonary veins (PVs).

Once the anatomical features are identified, a lesion path is chosen so as to surround the anatomical features. In one embodiment, the lesion path is chosen from among a catalog of available lesion paths, based on the location of the identified anatomical features. In some embodiments, a physician may prescribe the lesion path by drawing the lesion path around identified features. In various embodiments, a combination of lesion path selection techniques may be utilized, such as having the physician draw some lesion paths, and also selecting pre-defined paths. These drawing and selection options may be performed in various orders of sequence. Once the lesion path is chosen, the catheter is used to ultrasonically ablate the tissue along the lesion path and around the identified anatomical features.

Sensing and Ablation System.

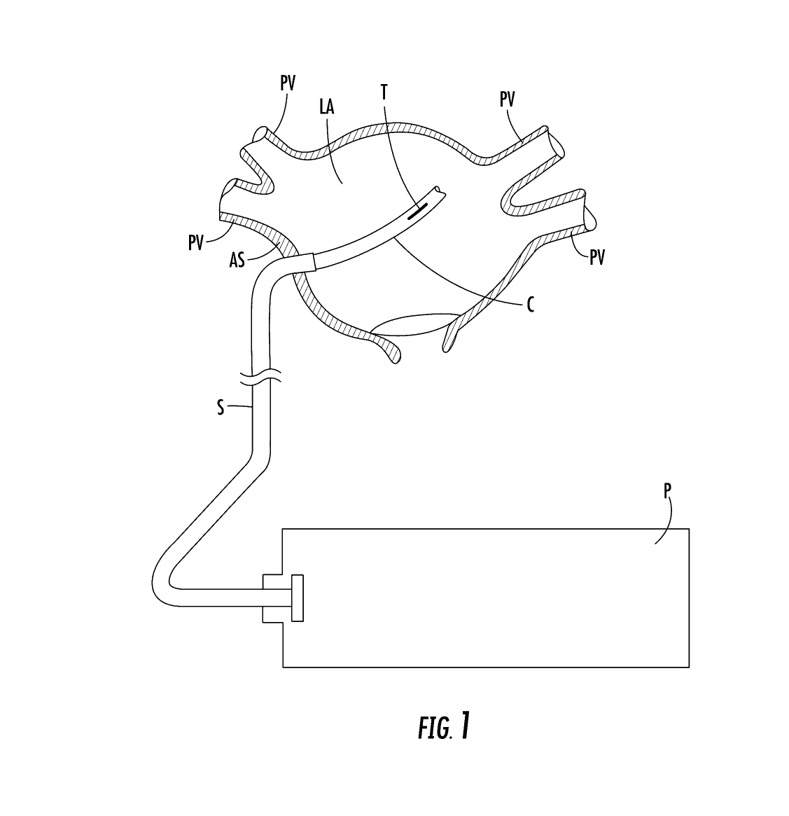

FIG. 1 is a diagrammatic illustration of an example catheter system for ultrasonically sensing and ablating tissue to treat atrial fibrillation, according to one embodiment. The system comprises a sensing and ablation catheter C transseptally disposed across an atrial septal wall AS into the left atrium LA of a patient's heart adjacent the pulmonary veins PV, and a console P outside of the patient. Console P, which may also be referred to as a controller, includes computer software and hardware for performing functions such as processing data collected by the Catheter C, and storing information related to the procedure. Catheter C comprises a proximal portion and a distal portion. The distal portion of the catheter C is configured for introduction into an atrium of the heart, either percutaneously or surgically, and comprises an ultrasonic transducer subassembly T (hereinafter also referred to as transducer T).

The transducer T is capable of ultrasonically sensing tissue, as well as ultrasonically ablating tissue, without necessarily establishing direct physical contact with tissue. That is, sensing of a region of tissue can be performed without requiring direct physical contact to be established with the surface of the tissue, although contact may occur due to, for example, motion of the catheter and/or tissue. The distal portion of the catheter C is configured to be moveable in a controlled fashion so that it may trace out sensing patterns and lesion paths. In one embodiment, and as shown in FIG. 1, the catheter device C is housed within a sheath S.

The catheter C may also include electromagnetic sensors (‘EM’ sensors, not shown) in the distal portion of the catheter, as well as along the shaft if desired, to provide positioning information during an ablation procedure. Detection of the EM sensors can be achieved by an EM positioning tracking system, such as by using a window field generator to track locations of the EM sensors. The EM sensors on catheter C may be 5 degree and/or 6 degree of freedom sensors. The use of two 5 degree of freedom EM sensors may be used in place of 6 degree of freedom sensors by using a unique software algorithm to determine roll correction after programmed movement of the catheter tip. In some embodiments, other types of position tracking systems can be utilized, such as those based on electrical impedance, fiber optics, and visual imaging (e.g. using radiopaque markers with monitoring of two orthogonal planes to track motion).

The console P is configured to couple to the proximal portion of the catheter C in order to direct the distal tip of catheter C to move in one or more directions, thereby guiding the transducer T along one or more sensing patterns or lesion paths. The console P also controls the operation of transducer T by delivering electrical energy to the transducer T in order to generate ultrasonic energy for sensing and ablating tissue, and by recording scan signals produced by transducer T as it senses the tissue surface.

As mentioned above, the console P controls the catheter C to move in a pattern, such as a raster pattern, in order to scan some portion of the tissue. Based on the received scan signals, console P then generates a 3-dimensional map of the tissue portion.

Based on the 3-dimensional map of the tissue portion, the console P presents one or more anatomical features, such as PVs, that are to be electrically isolated. The console P then suggests a lesion path based on the map and the location of the anatomical features, or a physician may select or prescribe the lesion path. Upon confirmation or modification of the lesion path by a user, the console P directs the catheter C to ablate the tissue along the lesion path.

In one embodiment, console P houses, or is coupled to, a memory element that stores a catalog of available lesion paths, from which catalog the lesion path is selected. The catalog may be configurable, and lesion paths may be added, deleted or modified. In one embodiment, the system further comprises a computer display or monitor in order to present the tissue map, the identified anatomical features, and the suggested lesion path to the user.

Additional details about the catheter C, transducer T, console P, and sheath S are disclosed in U.S. Patent Publication No. 2011/0257563, previously incorporated by reference. Other disclosure applicable to the ablation system described above is included in patent applications previously incorporated herein by reference.

Sensing Mode.

In operation, the transducer T functions in one of two modes: a sensing mode and an ablation mode. When operating in sensing mode, the transducer T is directed to move in a sensing pattern over a portion of atrial tissue surface, and to capture a set of ultrasonically generated data indicating the distance between the transducer T and the atrial tissue at a plurality of points along the traversed sensing pattern. In one embodiment, transducer T operates in Amplitude-mode (A-mode) to sense a distance between the transducer T and the tissue surface. The distance measurements can then be used to create images of the tissue, and to analyze characteristics of the tissue being treated.

Use of an A-mode for imaging is unique compared to conventional intravascular ultrasound techniques where Brightness-mode (B-mode) is used. B-mode typically utilizes a linear phased array of transducers to generate two-dimensional image slices, which can then be compiled or stacked up into a 3-D image. A conventional phased array forms an image slice by electronic beamforming using the multiple transducers. A-mode, however, takes point measurements based on echoes of the ultrasound waves reflected back from the tissue.

In the present disclosure, a 3-D map is created using data sensed by an ultrasound transducer in amplitude-mode. In some embodiments, data is collected for regions of the tissue, such as portions of a chamber of the heart, and then the regional maps are pieced together to form a combined surface map of a larger region. For example, the larger region may be a chamber of body tissue such as a chamber of the heart, e.g. the left atrium. The method includes sensing a first region of a chamber of body tissue, the sensing of the first region involving moving an ultrasound transducer of a catheter over a first surface of the first region along a first sensing pattern. The sensing of the first region also involves using the ultrasound transducer to gather a first set of echo-anatomical data in an amplitude mode at a first plurality of points along the first sensing pattern. The first set of echo-anatomical data includes distances between the ultrasound transducer and the first surface of the tissue at the first plurality of points. The method also includes sensing a second region of the chamber of body tissue, the sensing of the second region including moving the ultrasound transducer over a second surface of the second region along a second sensing pattern, and using the ultrasound transducer to gather a second set of echo-anatomical data in the amplitude mode at a second plurality of points along the second sensing pattern. The second set of echo-anatomical data includes distances between the ultrasound transducer and the second surface at the second plurality of points. A first three-dimensional surface map of the first region is generated using the first set of echo-anatomical data, and a second three-dimensional surface map of the second region is generated using the second set of echo-anatomical data. Note that although methods of combining region maps shall be described in this disclosure, multiple region maps are not required to perform the present ablation procedures. For example, ablation may be performed using a map created from only one mapping scan.

The first three-dimensional surface map and the second three-dimensional surface map are combined to form a combined surface map, as shall be described in more detail subsequently. In conventional magnetic resonance imaging (MRI) and computed tomography (CT) imaging, the volume data set is uniformly sampled in a grid. In the present disclosure, the surface maps are 3D surface reconstructions created by processing A-mode data from the ultrasound transducer to construct a non-uniform sampled volume data set. In some embodiments, the surface maps show the surfaces of the tissue, such as the endocardium and/or epicardium of a cardiac chamber in which the catheter is inserted, along with surfaces of vessels associated with the cardiac chamber. Although two regions are discussed in this example and elsewhere in this disclosure for forming a combined surface map, any number of “N” regions may be utilized, where N is ≧1. In some embodiments, the ultrasound transducer continuously moves during the sensing of the first region and the sensing of the second region.

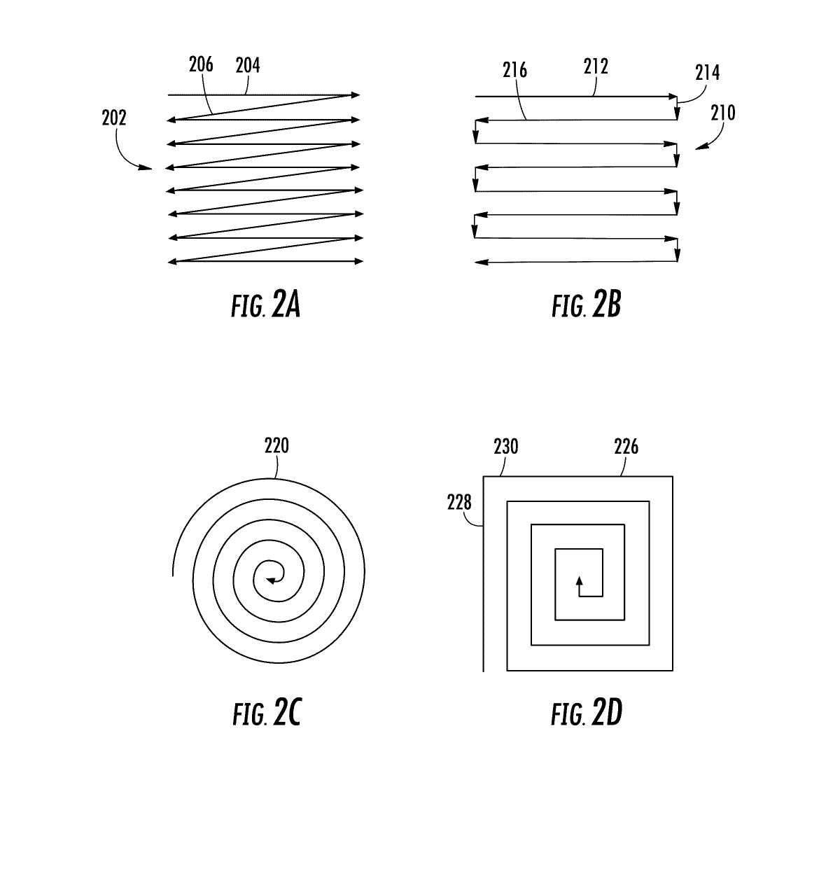

The sensing pattern may be a raster pattern, as shown in the examples of FIGS. 2A-2B. FIG. 2A illustrates a raster pattern 202 where the raster pattern scans horizontally from left to right 204 and a diagonal return 206 from right to left allows the next horizontal scan to begin again from left to right and the pattern is repeated multiple times. FIG. 2B illustrates a variation of a raster scan 210 in which scanning occurs horizontally 212 from left to right. At the end of the horizontal scan, the scan is vertically moved downward as indicated by arrow 214, and then the scan continues from right to left 216. Another vertical adjustment moves the scan downward again, and then the scan from left to right begins again. This pattern is repeated multiple times. FIGS. 2C-2D illustrate exemplary scan patterns having spiral shapes. For example, in FIG. 2C, the scan pattern has a curved pattern that spirals centrally inward to a central point, with each spiral having a smaller radius than the previous spiral. FIG. 2D illustrates a square spiral, where the scan pattern 226 has a series of vertical 228 and horizontal 230 scans that are joined together to form an inwardly directed square spiral. One of skill in the art will appreciate that the directions left, right, vertical and horizontal may be changed, and therefore are not intended to be limiting. In addition, while the illustrated raster and spiral patterns are provided as example shapes for sensing patterns, other geometries are possible and are covered in the scope of this disclosure. Furthermore, in some embodiments the user can manually create a sensing pattern such as via a free-form drawing mode, rather than choosing from pre-defined shapes stored in the system.

The sensed data is then used by the console P to generate a 3-dimensional surface map of the sensed portion of the atrial tissue. Thus the present system is useful for echo-anatomical mapping of the target tissue surface, such as a portion of, or the entire surface of the left or right atrium of the heart. The surface map may include the entire target treatment surface, or it may include only a section of the treatment surface. Because the catheter may require repositioning several times during mapping of the entire surface, it may be easier to map a section of the target surface, reposition the catheter, and then map another section. Also, in addition to positioning requirements, scanned sections may be limited to certain areas due to memory or data processing limitations of the system.

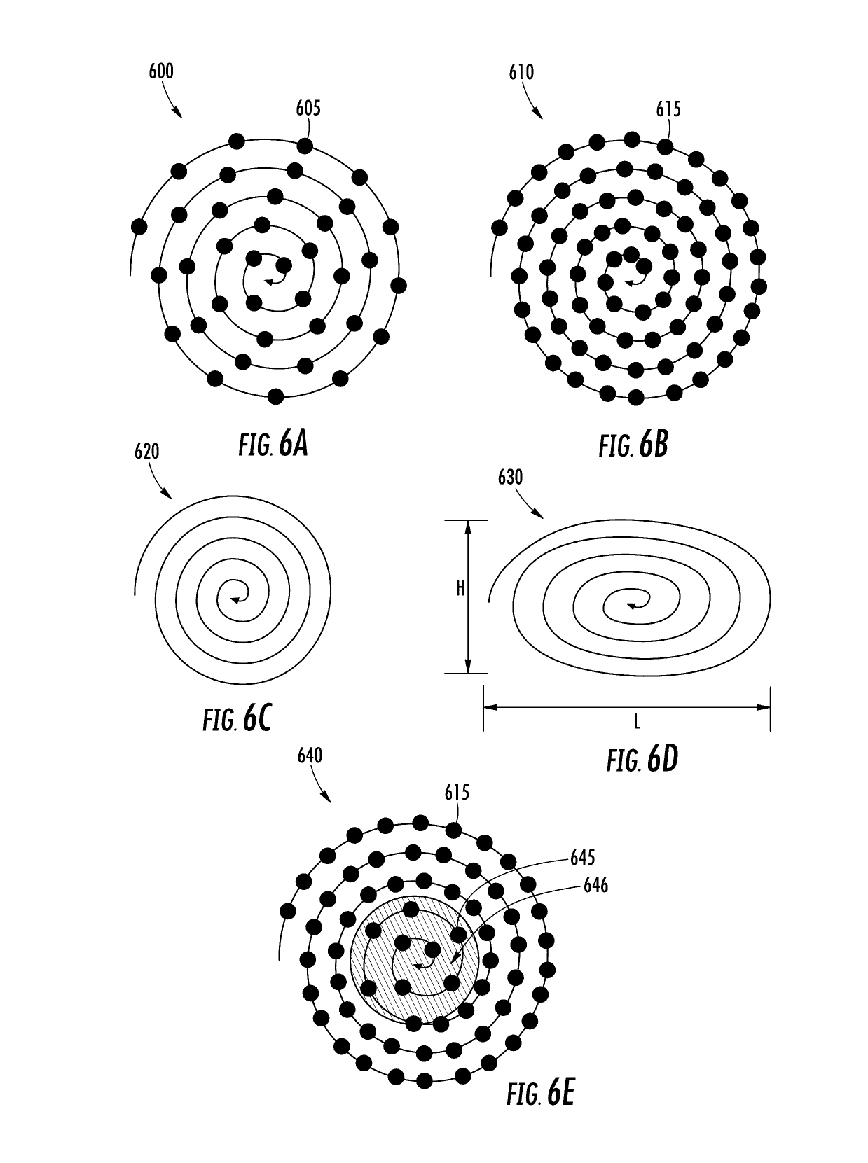

FIGS. 6A-6E illustrate further aspects of the sensing patterns, which may include pattern parameters that are adjustable by a user or by a console coupled to the catheter. Note that the patterns of FIGS. 2A-2D and FIGS. 6A-6E may be applied to the sensing of any region used to create a combined surface map, such as a first sensing pattern for a first region, a second sensing pattern of a second region, up to “N” regions. FIG. 6A shows a spiral sensing pattern 600 which is the same as that of FIG. 2D, but also shows a plurality of points 605 at which amplitude-mode measurements are taken. FIG. 6B illustrates a spiral pattern 610 which is identical to spiral pattern 600, but with an increased number of data points 615, resulting in an increase in the data density at which measurements are sensed. The data density may be increased by, for example, changing the acquisition rate at which measurements are taken, or by changing the speed at which the catheter moves along the sensing pattern. The data density enables the physician to customize the coarseness or fineness of the data collection.

In FIG. 6C, the spiral pattern 620 has been altered in size compared to spiral pattern 600, where pattern 620 has been proportionally scaled down in this example. The physician may desire to change the size of the pattern due to, for example, space constraints near a PV, or the particular geometry of the patient's anatomy. Note that in the example of FIG. 6C the number of spiral layers remains the same as in pattern 600, resulting in a greater data density. However, in other embodiments the size of the spiral pattern may be altered such that the spacing between spiral layers remains the same but the number of layers is increased for a larger size pattern, or decreased for a smaller size pattern. In FIG. 6D the spiral sensing pattern 630 has an aspect ratio that has been adjusted compared to spiral pattern 600, where the height H has been reduced and the length L has been increased. In other embodiments, the height and/or length may be increased, reduced, or remain constant in various combinations, and by various amounts. In the sensing pattern 640 of FIG. 6E, the data density of points 645 is intentionally reduced within a region 646 (for example, a pulmonary vein) compared to the data density of points 615 outside of region 646, by increasing the speed of the catheter motion to reduce the overall time required to generate a map. Reduced spatial sampling is less important inside a pulmonary vein since ablation lines are planned and delivered outside a pulmonary vein, and higher spatial sampling is desirable where the ablation paths are planned. This variable data density illustrated in FIG. 6E can be automatically controlled by the console, or can be user-defined.

Note that the adjusted parameters demonstrated by FIGS. 6A-6E are representative examples, and other variations are possible. For example, decreases in a parameter may be replaced by an increase, or vice versa. In addition, more than one of the parameters may be adjusted at the same time, such as a change in data density accompanied by a change in aspect ratio. Furthermore, the pattern parameter adjustments illustrated for a spiral pattern in FIGS. 6A-6E may be applied to other patterns as well, such as a raster pattern.

Adjustment of the pattern parameters—pattern size, data density and aspect ratio as illustrated by FIGS. 6A-6E—enables the sensing patterns to be optimized, such as for a specific anatomy and/or area being investigated. For example, in an area of high curvature of the tissue, higher resolution may be desired for more accurate planning of the lesion. In another example, the physician may wish to have more imaging resolution in an area near a pulmonary vein. Thus, the methods of the present disclosure provide the ability to sense a supplemental area in a region of tissue, such as a first region or second region, using the ultrasound transducer, where at least one of the pattern parameters is adjusted to increase a sensing resolution for the supplemental area. The supplemental area may be all of, or a portion of, the previously sensed regions; or may be a different region than previously sensed. In some embodiments, the user may adjust the parameters manually through a user interface (e.g., display, keyboard or other input device) connected to the console. In other embodiments, the console performs an analysis to identify regions of interest, and recommends a location of the supplemental area for supplemental sensing based on the regions of interest. In such embodiments, the console of the treatment system includes computer hardware and software to analyze the data collected by the ultrasound transducer, such as a first set of echo-anatomical data from a first region or a second set of echo-anatomical data a second region.

The analysis by the console to identify regions of interest for supplemental sensing can include, for example, searching for gradients that are above a certain threshold for distance measurements between the ultrasound transducer and tissue. In some embodiments, the console may highlight regions of a map using a unique color or other visual indicator where imaging resolution is not sufficient to support lesion planning.

Generating Maps.

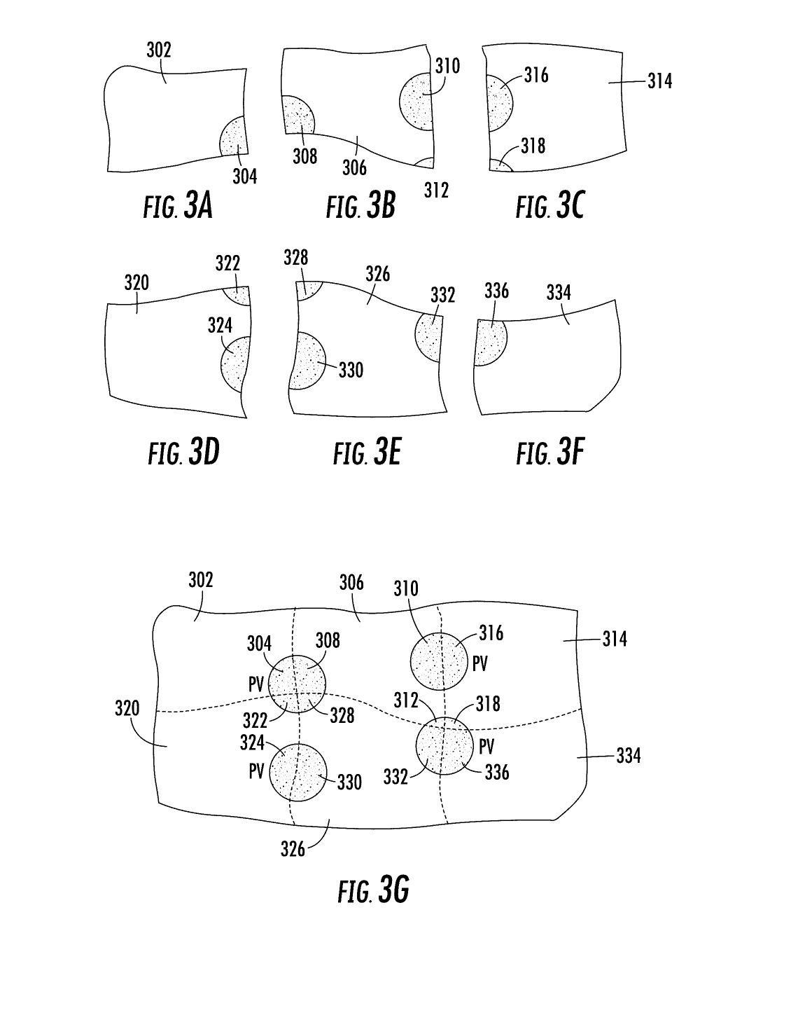

The process of sensing and obtaining scan signals in areas of the tissue with sensing patterns is repeated as needed in order to generate one or more further 3-dimensional maps for one or more neighboring portions of the atrial tissue surface, thereby covering the surface area that is to be mapped with sensing patterns. As one example, FIGS. 3A-3F show 3-dimensional maps for six neighboring portions of the atrial tissue surface having four pulmonary veins PV. FIG. 3A illustrates an upper left portion 302 of the target tissue and shows an upper left portion 304 of a first PV. FIG. 3B illustrates an upper center portion 306 of the target tissue and shows an upper right portion 308 of the first PV, an upper left portion 310 of a second PV and an upper left portion 312 of a third PV. FIG. 3C illustrates an upper right portion 314 of the target tissue and shows an upper right portion 316 of the second PV and an upper right portion 318 of the third PV. FIG. 3D illustrates a lower left portion 320 of the target tissue and shows a lower left portion 322 of the first PV and a lower left portion 324 of a fourth PV. FIG. 3E illustrates a lower center portion 326 of the target tissue and shows a lower right portion 328 of the first PV, a lower right portion 330 of the fourth PV and a lower left portion 332 of the third PV. FIG. 3F illustrates a lower right portion 334 of the target tissue and shows a lower right portion 336 of the third PV. The PVs are depicted as grey portions, indicating “holes” or regions of large distance between the transducer T and tissue. Once generated, these one or more 3-dimensional maps may be combined by the console P to form a combined 3-dimensional map of the scanned atrial tissue surface. FIG. 3G shows an example combined 3-dimensional map obtained by combining the six maps of FIGS. 3A-3F. Thus, the present system is capable of mapping a portion of, or mapping the entire inner surface of an atrium, or other tissue surface.

Note that in some applications of the present disclosure it may be determined that obtaining a single 3-dimensional map may be sufficient to allow identification of one or more PVs (instead of obtaining and combining a plurality of 3-dimensional maps, as described above). In the following description, the term “combined map” shall also refer to such a single 3-dimensional real time echo-anatomical map obtained in such embodiments. In some embodiments, the map is also compatible with other mapping and ablation systems, such as the CARTO® electroanatomical mapping system (Biosense Webster, Diamond Bar, Calif.), CT scanning systems, and the EnSite Array™ from St. Jude Medical, or other similar systems.

In some embodiments, the combined echo-anatomical map is used to identify the location of one or more PVs, which may appear as holes or similar artifacts on the map. The identification of the PV locations may be done algorithmically by the console P, or it may be done by a human user, or by using a combination of user input and programmed logic. Optionally, the echo-anatomical map may be presented to a user on a computer display in order to allow visual identification and/or visual verification of the PV locations.

In some embodiments, anatomical features are first identified in the maps of the regions, and the anatomical features are used to create a combined surface map from the region maps as illustrated in FIG. 3G. The console analyzes the first 3-dimensional surface map and the second 3-dimensional surface map for identifiable anatomical features. To detect identifiable anatomical features, the present approaches combine knowledge of point location based on the EM positioning system as a reasonable first approximation for registration. From the first approximation, areas with strong spatial gradients are mapped and rated against a feature overlap criteria. If sufficient overlap exists, a first point cloud (‘PC’) will be registered to the PC, spatial transformation, to find the best fit location. Points identified by feature identification may have more influence on establishing the transformation. The combined PC is processed to form a combined 3D map. Known techniques that may be utilized in these calculations include ICP (Iterative Closest Point) and RPM (Robust Point Matching). The identifiable anatomical features, if present, are used as reference points or datum features to combine the regions maps into the combined surface map. The identifiable anatomical feature may be, for example, a pulmonary vein or a left atrial appendage. In some cases, there may not be an identifiable/overlapping feature. When the console is unable to identify sufficient overlap with a common anatomical feature, the user will be notified. The user must either acquire additional data, which may require repositioning the catheter, or manually identify common features.

Use of Pre-Constructed Images.

In some embodiments, a pre-constructed image is utilized for identifying anatomical features, and the pre-constructed image can also be used to provide recommended sensing pattern parameters to the operator. In such embodiments, a pre-constructed image of the chamber of body tissue is obtained prior to the sensing of the tissue, such as sensing of a first region and a second region. This pre-constructed image may be an image generated in a previous procedure from data collected by the ultrasound transducer catheter of the present disclosure. In other embodiments, the pre-constructed image may be imported or loaded from an external system, such as computed tomography (CT) or magnetic resonance imaging (MRI). In some embodiments, the pre-constructed image may also include pre-procedure lesion plans.

Using the console, such with as computer software stored in memory elements and processed on processing hardware in the console or coupled to the console, one or more anatomical features in the pre-constructed image can be identified. Common anatomical features used for registration in the human left atrium include the PVs and the left atrial appendage (‘LAA’) ridge. PVs show high spatial gradients when moving along the surface of the posterior wall toward the vessel. A carina between two pulmonary veins is also a unique anatomical feature. The LAA ridge shows a distinct edge between the left PVs and the LAA. The LAA itself may also be a distance alignment region. Based on the anatomical features identified by the console, in some embodiments the console can provide recommended pattern parameters for the first sensing pattern, the second sensing pattern, and other sensing areas. The recommendations provide the ability to optimize the sensing patterns for aspects such as the presence of PVs or the specific geometry of the tissue in the patient being treated. One or more sensing patterns are displayed on the pre-constructed image, where the operator can view, accept, change, or delete the sensing patterns.

The pre-constructed image can also be used to identify anatomical features in the map created from the sensed ultrasound data. The identification of anatomical features involves registering—that is, correlating or aligning—the pre-constructed image with the ultrasound map using manual and/or automated techniques. In some embodiments, the user manually moves the pre-constructed image to closely align with the map that has been created with the scanned ultrasound data, and then an algorithm is used to automatically align the surfaces based on common anatomical features. The automatic approach is similar to the approach used to combine the first and second maps as described earlier. In various embodiments, user-assisted registration of anatomical landmarks can be utilized to roughly align the pre-constructed map. For example, the inferior vena cava and trans-septal locations can be marked as the ultrasound catheter is moved to the left atrium, to register the catheter to the pre-constructed image. In another example, the user can identify landmarks in the left atrium and also identify corresponding landmarks in the newly created ultrasound map, to assist the algorithm in the registration process.

Maps.

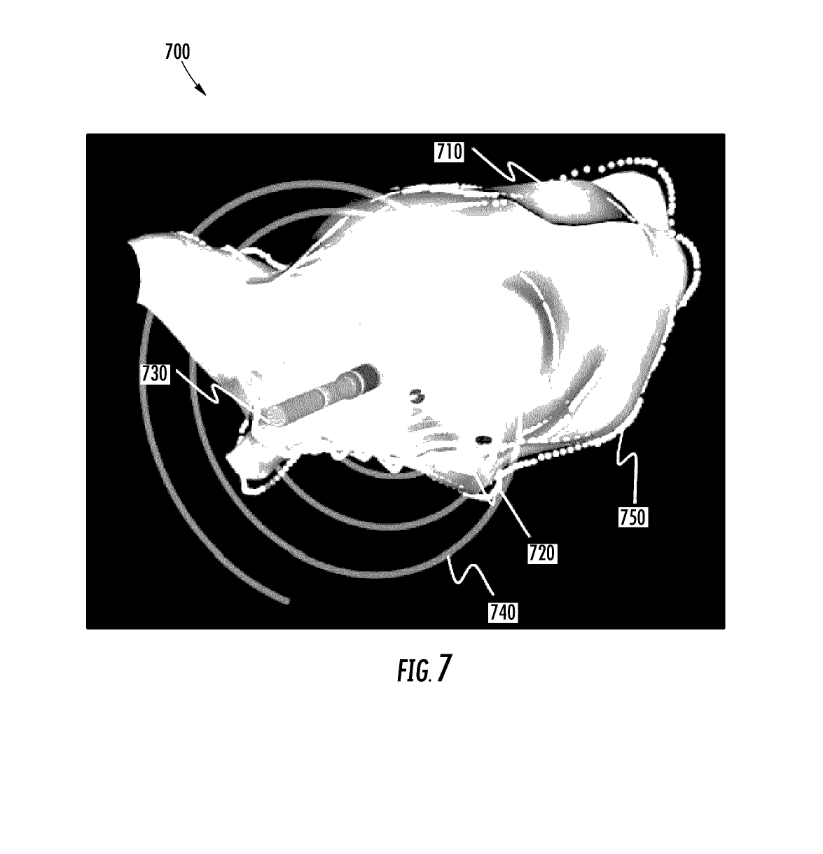

FIG. 7 illustrates an example 3D anatomical map generated from A-mode ultrasound data collected by the ultrasound catheter. The image 700 of FIG. 7 shows a reconstructed image of a left atrium 710, where an example lesion path 720 is superimposed on the image 700. A position of catheter 730 is also shown, where the catheter position has been registered with respect to the anatomy on the image. Also shown in image 700 is an example sensing pattern 740, and a point cloud 750 indicating discrete data points. Use of A-mode sensing with precise control of the ultrasound beam from the catheter tip in the present disclosure is a non-linear, piece-wise or sequential approach to creating a 3D map of the target volume. As the beam from the ultrasound transducer moves along a three-dimensional path (sensing pattern) gathering data, the totality of the transducer positions during the scan essentially forms a virtual array, where the array configuration is defined by the scan sensing pattern. Whereas in conventional B-mode imaging where the received ultrasound data is combined directly to form an image, the A-mode sensing data in the present disclosure is processed to form a set of points located in a 3D coordinate frame defining a point cloud (‘PC’). Each point is generated from detecting one or more tissue boundaries and other tissue characteristics. Tissue boundaries include the endocardial and epicardial surfaces in the heart, and can also include structures beyond the epicardium. Each point in the PC is defined by a 3D vector representing the location and pointing direction of the ultrasound beam. Adjacent points in the PC are combined to form a 3D image, resulting in a 3D surface reconstruction where surfaces can represent the endocardium, epicardium, including other structures, and other tissue characteristics. The methods of the present disclosure uniquely generate the PC from A-mode data, and apply unique filtering of the PC data prior to 3D surface reconstruction. The presentation of the PC and subsequent 3D surface are also unique via the constructed maps that are displayed, as shall be described in further detail below.

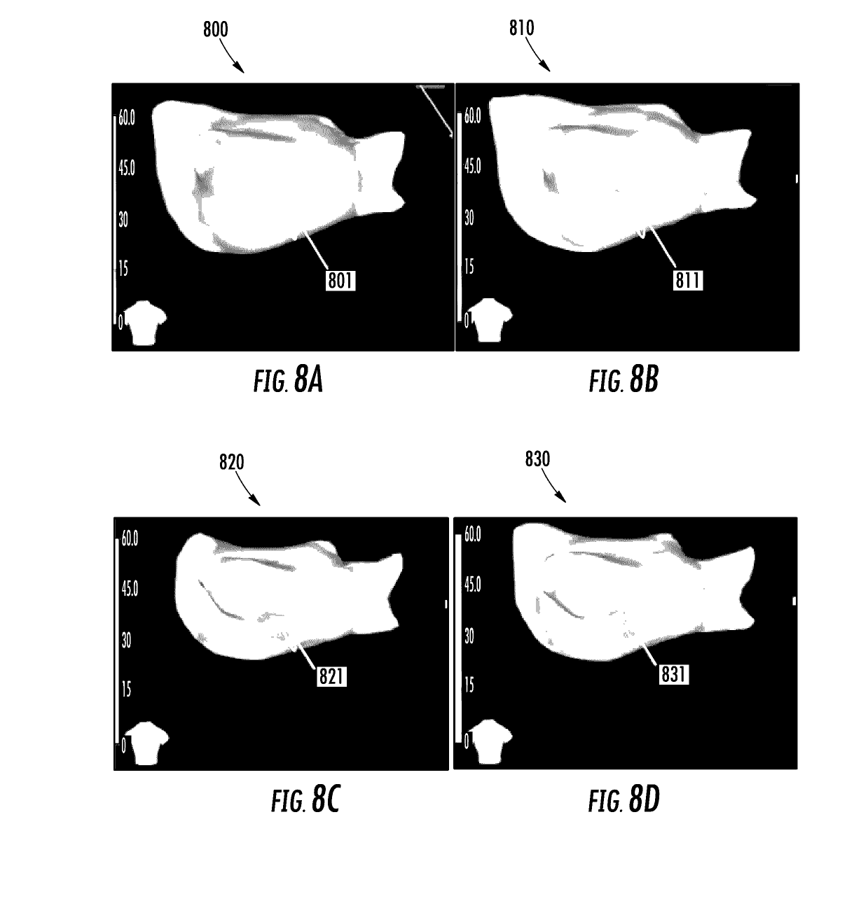

In some embodiments, three-dimensional image maps can be created that account for motion of the heart and/or catheter as illustrated in the family of motion maps of FIGS. 8A-8D. The ultrasound transducer gathers data, such as a first set of echo-anatomical data and a second set of echo-anatomical data for first and second regions in a cardiac chamber, over a plurality of cardiac and respiratory cycles. The combined map is formed by averaging the first set of echo-anatomical data and the second set of echo-anatomical data over the plurality of cardiac and respiratory cycles. In some embodiments, the combined surface map is a family of combined maps, where each map in the family of combined surface maps represents a point in time in the plurality of cardiac and respiratory cycles. For example, the images 800, 810, 820 and 830 of FIGS. 8A-8D represent four sequential time points in a cardiac cycle. The grayscale shading, which may also be shown as colors, in these images represent the distance from the catheter tip to the tissue. The darkest areas in the regions 801, 811, 821 and 831 indicate where the catheter tip is closest to the tissue. The color or shading gradient in the embodiments of FIGS. 8A-8D are for a scale of 0-60 mm, where the darkest regions represent distances less than 3 mm.

In further embodiments, motion of the catheter relative to motion of the body tissue is displayed on the family of combined maps. By characterizing absolute and relative tissue motion, the console is able to provide the user with a motion-compensated view of the tissue. This motion-compensated view provides a static view of the tissue with a live catheter display. The live catheter display depicts the realistic view of the relative motion between the catheter and the tissue. Showing relative motion presentation can decrease visual fatigue compared to showing the independent motion of the tissue and catheter. Additionally, the dynamics of relative motion are aggregated across the cardiac cycle and presented in a simplified form of a quality color map. The quality color map provides a summation of quality characteristics including, but not limited to, distance of the catheter tip to the tissue surface, angle of incidence of the ultrasound beam relative to the surface of the tissue, and tissue thickness across the cardiac cycle to help aid optimum catheter positioning and lesion planning.

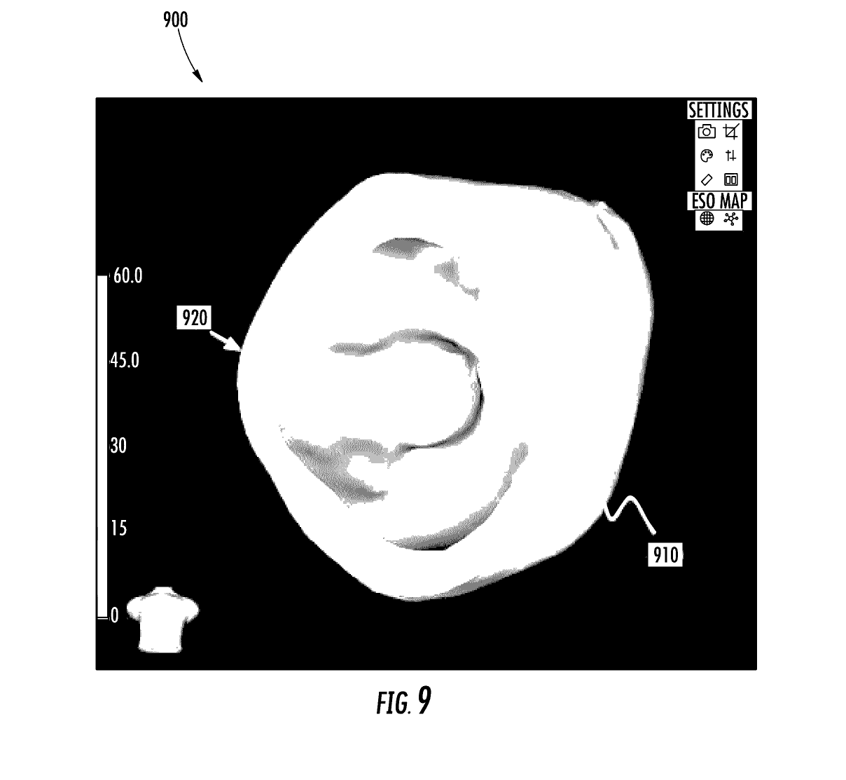

In some embodiments, tissue thickness maps are provided to assist in planning and monitoring an ablation lesion. FIG. 9 illustrates an example tissue thickness map 900 for the first sensing pattern or the second sensing pattern from the calculated tissue thicknesses. Tissue thicknesses are calculated along a sensing pattern, such as the first sensing pattern using the first set of echo-anatomical data, or the second sensing pattern using the second set of echo-anatomical data. Tissue thickness are calculated by first detecting the endocardial tissue surface, and second the epicardial tissue surface. The calculations are performed by a console of the treatment system, where the console includes computer software along with memory and processor hardware components to perform the calculations. Each of the endocardial and epicardial surfaces are generated from the data collected at the plurality of points along the sensing pattern (e.g., as described in relation to FIGS. 2A-2D and 6A-6E). In FIG. 9, the tissue thickness 920 is shown for a circumferential slice of the chamber 910. In some embodiments, the calculating includes normalizing the tissue thicknesses to account for angles of incidence during the sensing of the first region and the sensing of the second region. Tissue thickness for a given location on the endocardial surface is the distance from the endocardial point to the epicardial surface along a line normal to the endocardial surface location. This normalized tissue thickness is independent of the angle of incidence of the ultrasound beam direction. In contrast, the term effective tissue thickness in this disclosure is the distance from the endocardial surface to the epicardial surface along the ultrasound beam from the catheter tip. Tissue thickness maps can be created with colors or graphics to display the normalized tissue thicknesses as an alternative or additional means to the circumferential slice of the chamber 910 in FIG. 9.

Methods of Generating Maps.

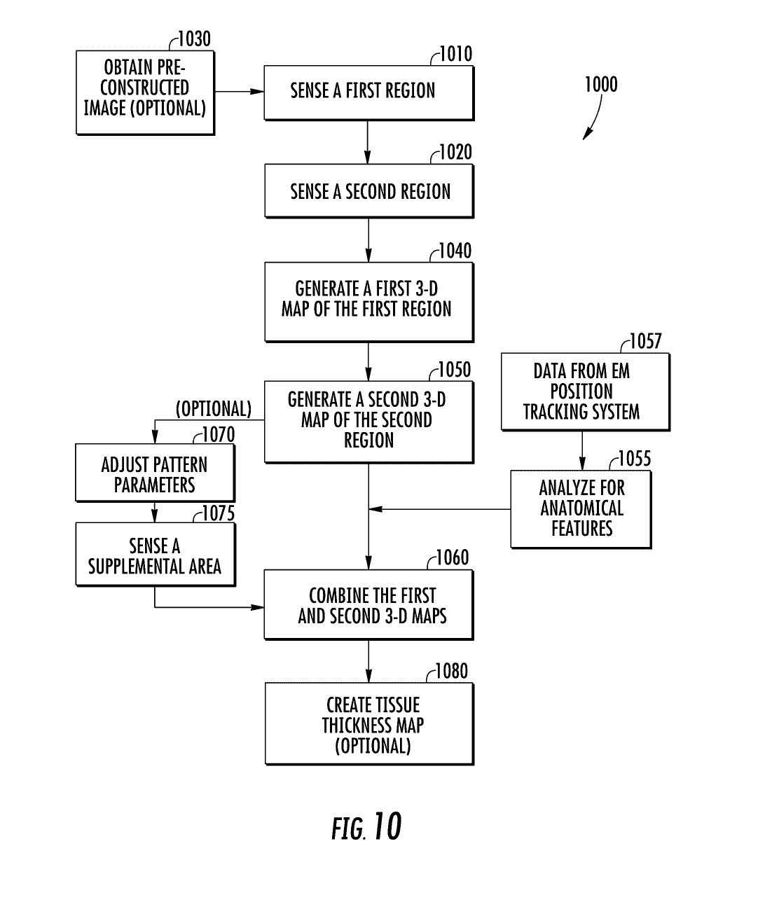

FIG. 10 is an example flow chart 1000 of methods of the present disclosure. In step 1010, a first region of a chamber of body tissue is sensed, using a catheter. The catheter has an ultrasound transducer, such as at the distal end of the catheter. A second region of the chamber of body tissue is sensed with the catheter in step 1020. The sensing of steps 1010 and 1020 involve moving the ultrasound transducer of the catheter over a surface of a region along a sensing pattern, and using the ultrasound transducer to gather a set of echo-anatomical data in an amplitude mode at a plurality of points along the sensing pattern. The set of echo-anatomical data includes distances between the ultrasound transducer and the surface at the plurality of points. In some embodiments, the sensing of the first region and the sensing of the second region are performed without requiring direct physical contact to be established with the first surface or the second surface. The ultrasound transducer may continuously move during the sensing of the first region and the sensing of the second region, in some embodiments. Optionally, a pre-constructed image, such as from an MRI or CT, can be obtained in step 1030 prior to sensing of the first and second regions.

Using data collected during the sensing of the first and second regions, a first 3-D map of the first region is generated in step 1040, and a second 3-D map of the second region is generated in step 1050. Step 1055 involves analyzing the echo-anatomical data from the sensing in steps 1010 and 1020 for identifiable anatomical features. The identifiable anatomical feature may be, for example, a pulmonary vein, a left atrial appendage, or a carina near pulmonary veins. The analysis of the maps for an identifiable anatomical feature can include selecting point locations in the first 3-dimensional surface map and the second 3-dimensional surface map using data from an electromagnetic position tracking system per step 1057; and comparing spatial gradients in the selected point locations against a feature overlap criteria.

The first and second maps are combined in step 1060 to form a combined surface map, using the identifiable anatomical features. The combined surface map of step 1060, in some embodiments, may include a family of surface maps that represent motion of the catheter and/or tissue, such as during different motion phases of the cardiac and respiratory cycles. The first set of echo-anatomical data and the second set of echo-anatomical data are gathered over a plurality of cardiac and respiratory cycles. The combined surface map is formed by averaging the first set of echo-anatomical data and the second set of echo-anatomical data over the plurality of cardiac and respiratory cycles. The combined surface map comprises a family of combined surface maps, wherein each map in the family of combined surface maps represents a point in time in the plurality of cardiac and respiratory cycles (i.e., a family of motion maps). The motion map family displays motion of the catheter relative to motion of the body tissue on the family of combined surface maps.

In some embodiments, pattern parameters of the sensing patterns can be adjusted in step 1070, such as to achieve a higher resolution in certain areas. That is, the first sensing pattern and the second sensing pattern have pattern parameters that are adjustable by a user or by a console coupled to the catheter, where the pattern parameters include a pattern size, a data density, and an aspect ratio. The adjusted pattern parameters are used to sense a supplemental area in step 1075. In some embodiments, the supplemental area is sensed in the first region or the second region using the ultrasound transducer, where at least one of the pattern parameters is adjusted to increase a sensing resolution for the supplemental area. In some embodiments, the console may be used to analyze the first set of echo-anatomical data or the second set of echo-anatomical data for regions of interest, and to recommend a location of the supplemental area based on the regions of interest. Prior to the sensing of the first region and the sensing of the second region, the methods may also include obtaining a pre-constructed image of the chamber of body tissue; registering a position of the catheter on the pre-constructed image; identifying, using the console, an anatomical feature in the pre-constructed image; providing recommended pattern parameters for the first sensing pattern and the second sensing pattern based on the anatomical feature identified by the console; and displaying the first sensing pattern and the second sensing pattern on the pre-constructed image.

Some embodiments also include creating a tissue thickness map in step 1080. The console calculates tissue thicknesses along the first sensing pattern using the first set of echo-anatomical data, or along the second sensing pattern using the second set of echo-anatomical data. A tissue thickness map is created for the first sensing pattern or the second sensing pattern from the calculated tissue thicknesses. The calculating can include normalizing the tissue thicknesses to account for angles of incidence of the ultrasound beam to the first surface of the tissue during the sensing of the first region and/or angles of incidence of the ultrasound beam to the second surface of the tissue during the sensing of the second region.

The methods of FIG. 10 can also include superimposing a lesion path onto the combined surface map, and ablating a target treatment tissue by applying a beam of ultrasound energy from the ultrasound transducer while moving the catheter along the lesion path in the chamber of body tissue. The ablating may be performed without requiring direct physical contact to be established with the target treatment tissue. The ablation procedure can include using a console to monitor deviations from the lesion path and to adjust movement of the catheter to correct the deviations.

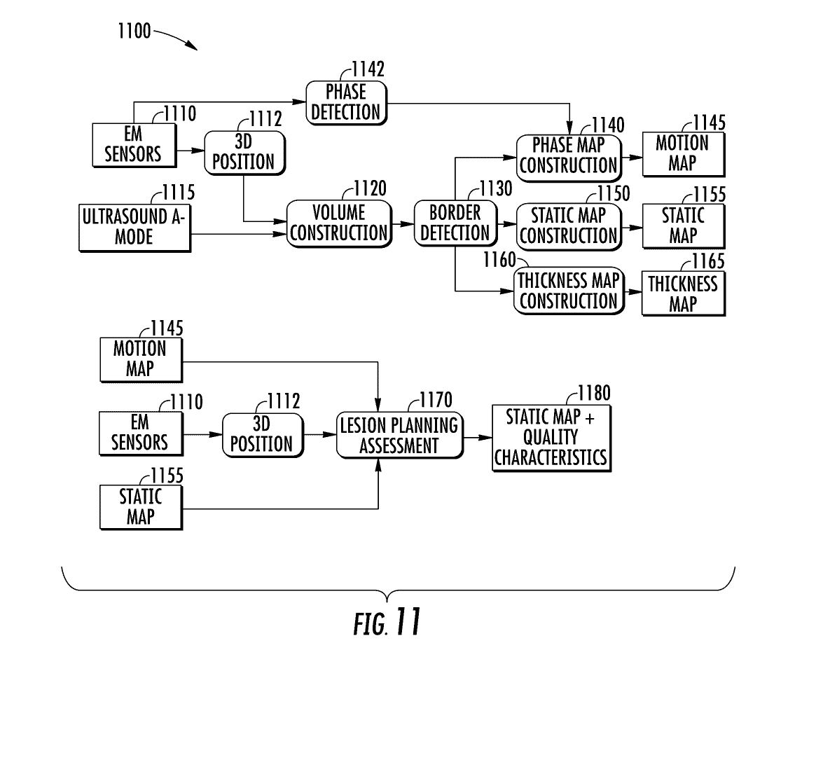

FIG. 11 is an example flow chart 1100 describing details for creating of a motion map family, a thickness map, and also static maps. Data from EM sensors 1110 provides 3D positions 1112 of the catheter tip, and the EM sensor data 1110 is used with ultrasound A-mode imaging data 1115 in a volume construction 1120. The volume construction 1120 is processed to detect endocardial and/or epicardial borders 1130 for each A-Mode sample and forms a set of 3D points (point cloud, or PC). Each PC is filtered, binned, based on the cardiac phase assigned to its associated A-Mode sample to generate a family of PC's, or N groups, in phase map construction 1140. Phase map construction 1140 also utilizes phase detection 1142 from EM sensors 1110, which results in motion map 1145. Endocardial and epicardial border detection 1130 is also used in static map construction 1150 to result in a static map 1155; and used in thickness map construction 1160 to result in a thickness map 1165. Motion map 1145, three-dimensional position data 1112 of the catheter tip from EM sensors 1110, and static map 1155 can also be utilized for a lesion planning assessment 1170 to provide a static map with quality characteristics 1180. Quality characteristics can be presented to the user for guiding lesion planning on maps where high quality lesions can be expected during ablation. Quality characteristics include tissue distances from the catheter tip that are within therapeutic ranges, angles of incidence that are preferred, and surfaces with high motion that are preferentially avoided for reliable lesion planning. Other quality characteristics can include tissue properties derived from processed ultrasound signals during map generation such as tissue density, tissue stiffness, and tissue compressibility. High or low values of these characteristics may guide preferred lesion plans.

In embodiments of FIG. 11, methods include sensing a region of a chamber of body tissue. The catheter has an ultrasound transducer, such as at a distal end of the catheter. The sensing includes moving the ultrasound transducer of the catheter over a surface of the region along a sensing pattern, and using the ultrasound transducer to gather a set of echo-anatomical data in an amplitude mode at a plurality of points along the sensing pattern. The catheter also includes electromagnetic sensors. The set of echo-anatomical data includes distances between the ultrasound transducer and the surface at the plurality of points. The set of echo-anatomical data is used to detect a border of the body tissue for each point in the plurality of points (border detection 1130), and also to detect motion phases (phase detection 1142) of the chamber of body tissue. The border of the body tissue is a surface boundary of the tissue, such as an epicardial border or an endocardial border, where one or both of the epicardial and endocardial borders may be detected. A three-dimensional surface map of the region is generated, being derived from the detected body tissue border and the detected motion phases of the region.

The method also includes displaying the three-dimensional surface map. The displayed three-dimensional surface map may be motion map 1145, having a family of multiple motion phase maps that are created from cycling through various motion phases (e.g. from phase map construction 1140). The motion phases can be phases in the cardiac and respiratory cycles. In other embodiments, the displayed three-dimensional surface map can be static map 1155. The static map may be, for example, quality map 1180 displaying quality characteristics, where the quality characteristics include one or more of: a distance between the ultrasound transducer and the surface of the tissue, an angle of incidence of the ultrasound beam to the surface, an amount of tissue motion, a tissue density, a tissue stiffness, and a tissue compressibility. The quality map may also have a lesion path superimposed onto it, where the method also includes ablating a target treatment tissue by applying a beam of ultrasound energy from the ultrasound transducer while moving the catheter along the lesion path in the chamber of body tissue.

The present disclosure also includes systems for performing the methods. The system can include a catheter having an ultrasound transducer, the ultrasound transducer configured to gather echo-anatomical data in amplitude mode along a sensing pattern. The catheter is coupled to a console which has computer hardware and software configured to receive, store, and process the data collected by the ultrasound transducer. Processing of the ultrasound data can include: generating three-dimensional surface maps, combining region maps to form combined surface maps, superimposing lesion paths onto the combined surface maps, monitoring deviations from the lesions path, adjusting movement of the catheter to correction the deviations, analyzing the echo-anatomical data for regions of interest, recommending a location of a supplemental area for sensing based on the regions of interest, calculating tissue thicknesses. The system can also be configured to obtain a pre-constructed image of the chamber of body tissue, register a position of the catheter on the pre-constructed image, identify an anatomical feature in the pre-constructed image, provide recommended sensing pattern parameters, and displaying the sensing patterns on the pre-constructed image. The console can also be configured to analyze region maps for identifiable anatomical features to use forming the combined surface map. The system can also include an electromagnetic position tracking system, such as one or more EM sensors on the catheter and a field generator to track the locations of the EM sensors. The system can also include a display on which the various maps and ablation procedure information are displayed. Maps with quality characteristics can also be displayed to guide preferred lesion planning.

Lesion Paths.

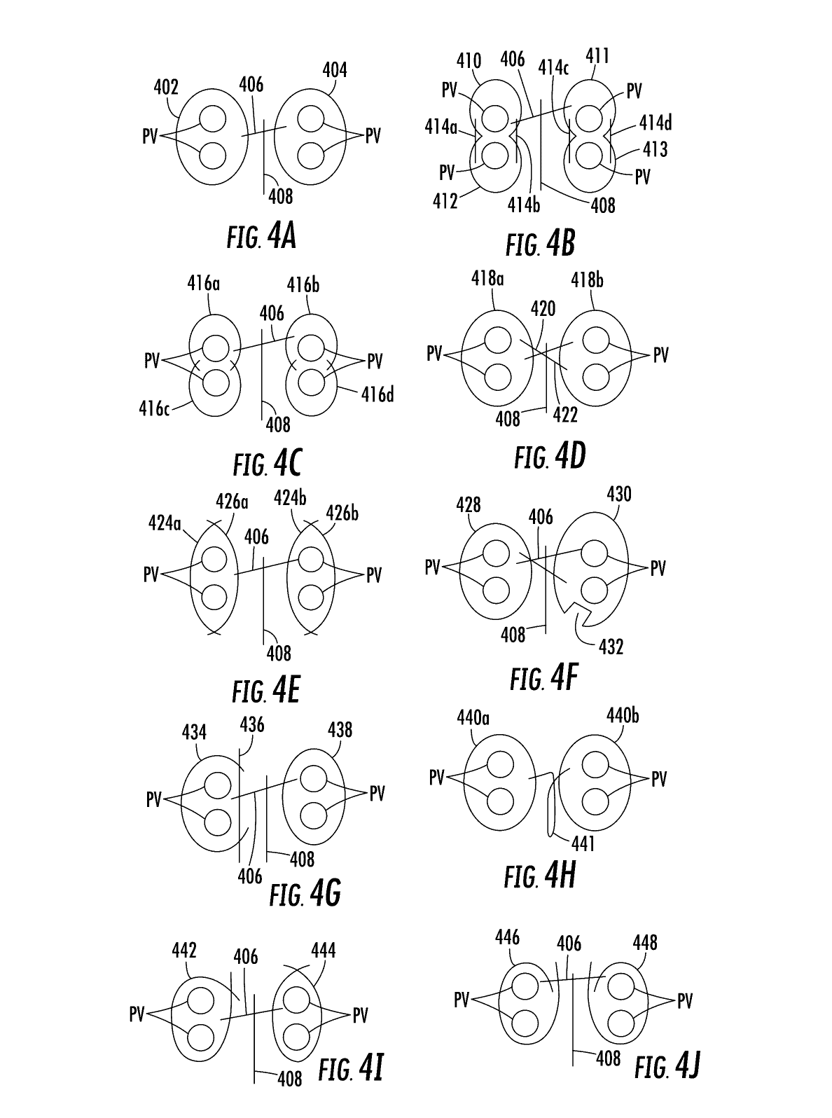

Once the PVs are located, a lesion path is selected from among the catalog of available lesion paths. FIGS. 4A-4J show example lesion paths in a catalog of lesion paths. FIG. 4A illustrates oval or circular lesions 402, 404 encircling two pulmonary veins each (e.g. two left pulmonary veins and two right pulmonary veins). A linear lesion 406 connects each of the oval lesions 402, 404, and a transverse lesion 408 extends from the linear lesion 406 downward toward the mitral valve (not illustrated). FIG. 4B illustrates another embodiment where an arcuate lesion 410 which is U-shaped, or horseshoe shaped, is superior to, and partially encircles a first upper PV, and a second arcuate lesion 411, that may take the same form as lesion 410, is superior to, and partially encircles a second upper PV. An arcuate lesion 412, which is U-shaped, or horseshoe-shaped is inferior to, and partially encircles a third PV, and another arcuate lesion 413 that may take the same form as lesion 412, is inferior to, and partially encircles a fourth PV. In this example embodiment, the first PV is superior to the third PV and the fourth PV is inferior to the second PV. Also, in this example embodiment, the first and third PVs are disposed to the left of the second and fourth PVs. Thus, some of the PVs may be left pulmonary veins, and some of the PVs may be right pulmonary veins. Linear lesions 414a, 414b connect the superior arcuate lesion 410 with the inferior lesion 412 so that the first and third PVs are completely encircled. Linear lesions 414c, 414d connect the superior arcuate lesion 411 with the inferior lesion 413 so that the second and fourth PVs are completely encircled. Linear lesion 406 connects the lesions encircling the pairs of PVs, and a transverse lesion 408 extends from the linear lesion 406 downward toward the mitral valve (not illustrated).

FIG. 4C illustrates still another embodiment where lesions 416a, 416b, 416c, and 416d arc around each of four PVs, such that two pairs of arcs 416a, 416c, and 416b, 416d merge together such that each pair completely encircles two PVs. A horizontal lesion 406 and a transverse lesion 408 connect the lesions encircling two PVs. FIG. 4D illustrates yet another embodiment of a lesion pattern where two oval or circular lesions 418a, 418b each completely encircle two PVs. Two linear lesions 420, 422 join the two oval lesions 418a, 418b forming an “X.” A transverse lesion 408 extends from the “X” downward toward the mitral valve (not shown). FIG. 4E illustrates another embodiment of a lesion where two arcs 424a, 426a completely encircle two PVs. The first arc 424a partially encircles one side of the pair of PVs, and the second arc 426a partially encircles the opposite side of the pair of PVs. The ends of the two arcs 424a, 426a crossover or intersect with one another to form a closed loop. Similarly, another pair of arcs 424b, 426b completely encircle a second pair of PVs. The third arc 424b partially encircles one side of the second pair of PVs, and the fourth arc 426b partially encircles the opposite side of the second pair of PVs. The ends of the third and fourth arcs 424b, 426b crossover one another or intersect with one another to form a closed loop. The pattern also includes a linear lesion 406 and a transverse lesion 408 that generally take the same form as previously described. FIG. 4F illustrates another lesion pattern having an oval or circular lesion 428 encircling two PVs. A second oval or circular lesion 430 encircles another two PVs, and also has a square or rectangular notch 432 to exclude the notched region from being encircled by the lesion. A linear lesion 406 connects the two lesions 428, 430, and a transverse lesion 408 extends therefrom.

FIG. 4G shows another example lesion pattern with an arc 434 partially encircling two PVs and a linear lesion 436 crossing both ends of the arc 434 so that the resulting lesion completely encircles both PVs. A second oval or circular lesion 438 completely encircles two other PVs, and a linear lesion 406 connects the two lesions 436, 438. A transverse lesion 408 extends from the linear lesion 406. FIG. 4H shows another example lesion having a curved lesion 441 connecting two circular or oval lesions 440a, 440b each encircling two PVs. The curved lesion 441 has two ends that overlap with each of the oval lesions 440a, 440b, and the curved lesion also overlaps itself, forming a lower loop similar to the Greek letter gamma. FIG. 4I illustrates a first loop 442 that encircles two PVs, and a second loop 444 that encircles two additional PVs. Each loop 442, 444 has overlapping ends such that the two PVs are completely encircled. A linear lesion 406 connects the two loops 442, 444 and a transverse lesion 408 extends from the linear lesion 406. FIG. 4J shows another embodiment where loops 446, 448 encircle two PVs each. However, in this embodiment, the ends of the loops 446, 448 do not overlap with one another and thus, while the PVs are completely encircled, a total conduction block has not been created, as the aberrant electrically activity can pass between the ends of the loops which do not overlap. Therefore, a linear lesion 406 extends through the open portions of each loop, and between both loops 446, 448, creating the conduction block. A transverse lesion 408 extends from the linear lesion 406.

The catalog of ablation patterns may be stored on a memory element coupled to the console P, or otherwise be made accessible to the console P. The choice of the particular lesion path to be used for ablation is based on the identified locations of the PVs in the combined 3-dimensional map of the atrial tissue, with the lesion path chosen to surround the PVs in order to electrically isolate them and thereby treat atrial fibrillation.

In one embodiment, the console P may be programmed to suggest a lesion path based on image analysis techniques applied to the obtained tissue map in order to locate artifacts, such as holes or ovals, which indicate the location of PVs. The user (for example, a surgeon or physician) may then accept the suggested lesion path, modify the suggested lesion path, choose another lesion path from the catalog, or draw a new lesion path. In such an embodiment, the console P may superimpose the selected lesion path onto the obtained surface map and present them to the user, thereby allowing the user to make any needed modifications prior to ablation. In some embodiments, the present methods include superimposing a lesion path onto the combined surface map, as illustrated in FIG. 7. The map may have been generated from ultrasound data collected by the catheter, or may be a pre-constructed image. The lesion path can be drawn by the user, suggested by the console P, or be a path that is modified by the user based on a suggested path from the console. The physician can ablate a target treatment tissue with the superimposed lesion path viewable on the combined surface map, by applying a beam of ultrasound energy from the ultrasound transducer while moving the catheter along the lesion path in the chamber of body tissue.

Additionally and optionally, the console P may be configured to learn from the user's (i.e., surgeon's or physician's) input with respect to lesion choices and lesion path modifications, by storing such information and associating it with the corresponding tissue maps and identified PV locations, for future reference. This allows the console P to personalize lesion path choices to particular surgeons, to suggest lesion paths based on past choices aggregated over a number of surgeons, etc.

Additional details on sensing and mapping may be found in U.S. Pat. Nos. 9,033,885; 8,414,508; and 9,192,789, each previously incorporated herein by reference. Other details which may be applicable are disclosed in other patent applications previously incorporated herein by references.

Ablation Mode.

Once a lesion path is chosen, the console P causes the transducer T to switch to operating in ablation mode. In ablation mode, the electrical energy delivered to the transducer T, and therefore the ultrasonic energy delivered by the transducer T to the tissue, is higher than in sensing mode, and sufficient to ablate the tissue. In this mode, the console P directs the catheter C to move the transducer T along the chosen lesion path while the transducer T ultrasonically ablates atrial tissue along the chosen lesion path, thereby creating an ablation lesion around the one or more PVs. The ablating may be performed without requiring direct physical contact to be established with the target treatment tissue. That is, contact between the ultrasound transducer and tissue is not needed in order for delivery of the ultrasound energy to occur. However, contact between the ultrasound transducer and tissue may occur due to, for example, movement of the tissue being treated or movement of the catheter as it is being advanced along the lesion path. In some embodiments, the console may provide a visual or audible notification that the transducer is in contact with the tissue.

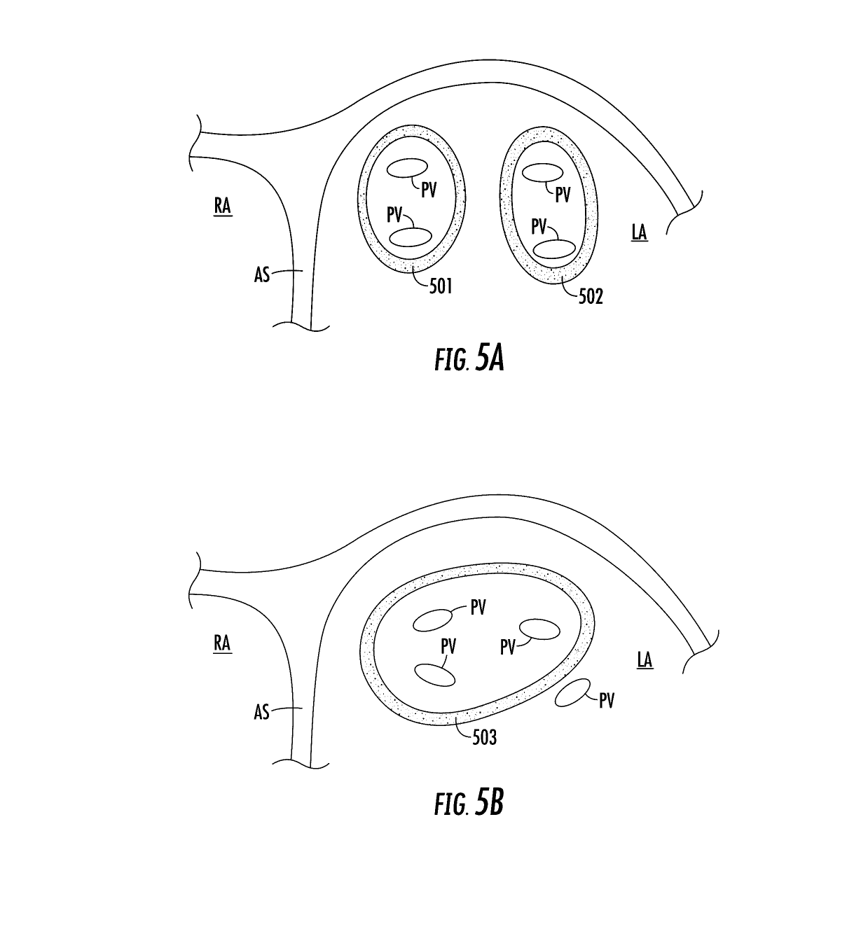

FIGS. 5A and 5B show example lesions created in the left atrium LA of the heart. In this embodiment, the left atrium LA has four pulmonary veins PV. The left atrium is separated from the right atrium via an atrial septal wall AS. FIG. 5A shows an example lesion 501 created around two PVs and lesion 502 created around another two PVs. Both lesions 501, 502 may be circular, elliptical, oval, or another shape (e.g. square, rectangular, etc.) and completely encircle two PVs. FIG. 5B shows an example lesion 503 created around three PVs in the left atrium LA. The lesion 503 may be circular, oval, elliptical, or any other shape (e.g. square, rectangular, etc.) that completely encircles the three PVs. In an optional embodiment, the console P may be configured to monitor deviations from the chosen lesion path and to provide corrections by adjusting the movement of the catheter C. For example, the console P may be configured to monitor a distance between the chosen lesion path and the tissue site that is being ultrasonically ablated by the transducer T, and move the distal portion of the catheter C (and with it the transducer T) towards the chosen lesion path in order to decrease that distance, thereby repositioning the transducer T along the chosen lesion path. In another optional embodiment, the console P may be configured to detect the transducer's T passing over veins and provide a visual indication thereof (for example, by flashing a red light when going over a vein and a green light otherwise), thereby giving an opportunity to the surgeon to intervene or to provide corrections at a later time.

Additionally and optionally, the console P may be configured to synchronize the operation of the transducer T, in sensing mode and/or in ablation mode, with the cardiac cycle. This is to enable greater accuracy in sensing and/or in ablation given the beating of the heart. Such synchronization may be accomplished by configuring the console P to receive input from a monitoring device such as an electrocardiograph (EKG), a computed tomography (CT) scanner, an electroanatomical mapping system (CARTO), or other such devices. The operation of the transducer T is then synchronized to accommodate or better account for the movement of the heart. For example, the console P may synchronize with the cardiac cycle and cause the transducer T to operate within periodic time slices in the cardiac cycle where the movement of the heart tissue is at a minimum, such as during physical diastole when the heart is stationary for the longest period of time during the cardiac cycle.

Additionally and optionally, the console P may be programmed to analyze the scan signals, received from the transducer T in sensing mode, and infer information about the thickness of the produced ablation. For example, this may be accomplished by comparing the times at which various tissue boundaries reflect the ultrasound emitted by the transducer T, and inferring the distance between such tissue boundaries (i.e., the thickness of the tissue between the boundaries). When applied to the two tissue boundaries on each side of an ablated layer, the ablation thickness may be inferred. Such thickness values may be used to more accurately time the exposure of atrial tissue to ultrasonic ablation energy, thereby providing for substantially transmural ablation and electrical isolation of the PVs.

While the specification has been described in detail with respect to specific embodiments of the invention, it will be appreciated that those skilled in the art, upon attaining an understanding of the foregoing, may readily conceive of alterations to, variations of, and equivalents to these embodiments. For example, different sets of islands could serve the charge seeding and charge removal purposes described above in the same DBD discharge volume. These and other modifications and variations to the present invention may be practiced by those skilled in the art, without departing from the scope of the present invention, which is more particularly set forth in the appended claims.

What is claimed is:

1. A method comprising:
sensing a first region of a chamber of body tissue with a catheter, the catheter comprising an ultrasound transducer, and the sensing of the first region comprising: a) moving the ultrasound transducer of the catheter over a first surface of the first region along a first sensing pattern, and b) using the ultrasound transducer to gather a first set of echo-anatomical data in an amplitude mode at a first plurality of points along the first sensing pattern, the first set of echo-anatomical data comprising distances between the ultrasound transducer and the first surface at the first plurality of points;
sensing a second region of the chamber of body tissue, the sensing of the second region comprising: c) moving the ultrasound transducer over a second surface of the second region along a second sensing pattern, and d) using the ultrasound transducer to gather a second set of echo-anatomical data in the amplitude mode at a second plurality of points along the second sensing pattern, the second set of echo-anatomical data comprising distances between the ultrasound transducer and the second surface at the second plurality of points;
generating, using the first set of echo-anatomical data, a first three-dimensional surface map of the first region;
generating, using the second set of echo-anatomical data, a second three-dimensional surface map of the second region; and
combining the first three-dimensional surface map with the second three-dimensional surface map to form a combined surface map.

2. The method of claim 1, wherein the sensing of the first region and the sensing of the second region are performed without requiring direct physical contact to be established with the first surface or the second surface.

3. The method of claim 1, further comprising:
superimposing a lesion path onto the combined surface map; and
ablating a target treatment tissue by applying a beam of ultrasound energy from the ultrasound transducer while moving the catheter along the lesion path in the chamber of body tissue.

4. The method of claim 3, wherein the ablating is performed without requiring direct physical contact to be established with the target treatment tissue.

5. The method of claim 3, further comprising using a console to monitor deviations from the lesion path and to adjust movement of the catheter to correct the deviations.

6. The method of claim 1, wherein the ultrasound transducer continuously moves during the sensing of the first region and the sensing of the second region.

7. The method of claim 1, wherein the first sensing pattern and the second sensing pattern comprise pattern parameters that are adjustable by a user or by a console coupled to the catheter, the pattern parameters selected from the group consisting of: a pattern size, a data density and an aspect ratio.

8. The method of claim 7, further comprising:
sensing a supplemental area in the first region or the second region using the ultrasound transducer, wherein at least one of the pattern parameters is adjusted to increase a sensing resolution for the supplemental area.

9. The method of claim 8, further comprising:
analyzing, using the console, the first set of echo-anatomical data or the second set of echo-anatomical data for regions of interest; and
recommending, using the console, a location of the supplemental area based on the regions of interest.

10. The method of claim 7, further comprising, prior to the sensing of the first region and the sensing of the second region:
obtaining a pre-constructed image of the chamber of body tissue;
registering a position of the catheter on the pre-constructed image;
identifying, using the console, an anatomical feature in the pre-constructed image;
providing recommended pattern parameters for the first sensing pattern and the second sensing pattern based on the anatomical feature identified by the console; and
displaying the first sensing pattern and the second sensing pattern on the pre-constructed image.

11. The method of claim 1, further comprising:
analyzing, using a console, the first three-dimensional surface map and the second three-dimensional surface map for an identifiable anatomical feature; and
using the identifiable anatomical feature in the combining to form the combined surface map.

12. The method of claim 11, wherein the identifiable anatomical feature is a pulmonary vein, a left atrial appendage, or a carina near the pulmonary vein.

13. The method of claim 11, wherein the analyzing comprises:
selecting point locations in the first three-dimensional surface map and the second three-dimensional surface map using data from an electromagnetic position tracking system; and
comparing spatial gradients in the selected point locations against a feature overlap criteria.

14. The method of claim 1, wherein the first set of echo-anatomical data and the second set of echo-anatomical data are gathered over a plurality of cardiac and respiratory cycles.

15. The method of claim 14, wherein the combined surface map is formed by averaging the first set of echo-anatomical data and the second set of echo-anatomical data over the plurality of cardiac and respiratory cycles.

16. The method of claim 14, wherein the combined surface map comprises a family of motion maps, wherein each motion map in the family of motion maps represents a point in time in the plurality of cardiac and respiratory cycles.

17. The method of claim 16, further comprising:
displaying motion of the catheter relative to motion of the body tissue on the family of motion maps.

18. The method of claim 1, further comprising:
calculating tissue thicknesses along i) the first sensing pattern using the first set of echo-anatomical data or ii) the second sensing pattern using the second set of echo-anatomical data, the calculating being performed by a console; and
creating a tissue thickness map for the first sensing pattern or the second sensing pattern from the calculated tissue thicknesses.

19. The method of claim 18, wherein the calculating comprises normalizing the tissue thicknesses to account for angles of incidence of the ultrasound beam to the first surface and the second surface during the sensing of the first region and the sensing of the second region.

20. A method comprising:
sensing a region of a chamber of body tissue with a catheter, the catheter comprising an ultrasound transducer and electromagnetic sensors, and the sensing of the region comprising: a) moving the ultrasound transducer of the catheter over a surface of the region along a sensing pattern, and b) using the ultrasound transducer to gather a set of echo-anatomical data in an amplitude mode at a plurality of points along the sensing pattern, the set of echo-anatomical data comprising distances between the ultrasound transducer and the surface at the plurality of points;
detecting, using the set of echo-anatomical data, a border of the body tissue for each point in the plurality of points, the border comprising at least one of an epicardial border and an endocardial border;
detecting, using the set of echo-anatomical data, motion phases of the chamber of body tissue;
generating a three-dimensional surface map of the region from the detected border of the body tissue and the detected motion phases of the region; and
displaying the three-dimensional surface map.

21. The method of claim 20, wherein the displayed three-dimensional surface map comprises a family of motion maps that can be cycled through the motion phases.

22. The method of claim 20, wherein the displayed three-dimensional surface map is a static map.

23. The method of claim 22, wherein the static map is a quality map comprising a displayed quality characteristic, wherein the displayed quality characteristic is selected from the group consisting of: the distances between the ultrasound transducer and the surface, an angle of incidence of the ultrasound beam to the surface, an amount of tissue motion, a tissue density, a tissue stiffness, and a tissue compressibility.

24. The method of claim 23, further comprising:
superimposing a lesion path onto the quality map; and
ablating a target treatment tissue by applying a beam of ultrasound energy from the ultrasound transducer while moving the catheter along the lesion path in the chamber of body tissue.
